SEQUENCE LISTING (1 sequence)

SEQ ID NO: 1 - DNA, 12 bp, Artificial Sequence
  Description of Artificial Sequence Synthetic polynucleotide sequence
agtctcgatcga

(19) United States
(12) Patent Application Publication
(10) Pub. No.: US 2005/0208199 A1
(43) Pub. Date: Sep. 22, 2005

(54) Methods and compositions for synthesis of 3'-aminolinkers

(76) Inventors: Julia Golova, Willowbrook, IL (US); Boris Chernov, Willowbrook, IL (US); Alexander Kukhtin, Lockport, IL (US)

Correspondence Address: BARNES & THORNBURG, P.O. BOX 2786, CHICAGO, IL, 60690-2786, US

(21) Appl. No.: 10/803,280

(22) Filed: Mar. 18, 2004

(51) Int. Cl. C12M 1/34; C07H 21/04

(52) U.S. Cl. 427/2.11; 536/23.1; 435/287.2

(57) ABSTRACT

Methods and compositions for 3′-aminomodifier CPG and 3′-aminomodified oligonucleotides are disclosed. Microarrays or biochips with 3′-aminomodified oligonucleotides are disclosed. A variety of detectable labels can be attached to 3′-aminomodified oligonucleotides.

The United States Government has rights in the disclosed invention pursuant to Contract No. W-31-109-ENG-38 between the U.S. Department of Energy (DOE) and the University of Chicago representing Argonne National Laboratory.

### BACKGROUND

Oligonucleotides used as DNA probes on supports, e.g., DNA-based microarrays, generally contain a 3′- or 5′-modification for immobilization on the support. Covalent immobilization (attachment) of such modified probes (e.g., having an aminoalkyl functional group attached to 3′- or 5′-end through a linker) to supports resulting from the reaction of amino groups linked to the modified probes with functional groups found on the supports, such as the reductive amination of carbonyl or reaction with activated carboxylic function. Introduction of modifications at the 3′-end of an oligonucleotide for further immobilization of probes on supports has become a useful tool in DNA-based microarrays manufacturing, which is now widely used fundamental science by researchers , for the detection of pathogens, viruses and bacterial species and also in diagnostics. Such modification of oligonucleotides including the introduction of an amino group attached at the 3′-end of the oligonucleotide through a linker can be used to post-synthetically introduce a variety of further modifications like different kinds of fluorophores, biotin and other chemical or biological moieties as diagnostic labels. However, the present commercially available 3′-aminomodifiers are restricted in their reactivity and are expensive.

### SUMMARY

Methods and compositions that include 3′-aminoterminal linkers and 3′-aminoterminal linker oligonucleotide conjugates are disclosed. The conjugates may be used to synthesize oligonucleotides on solid supports.

3′-aminoalkyl modification is produced during chemical synthesis of oligonucleotides by using a 3′-amino modifier. In an embodiment, the modifier represents a polyfunctional chemical that is a donor of an amino group and is also connected with a polymer support such as, for example, controlled pore glass (CPG). Other supports such as glass or plastic slides can also be used. The aminoalkyl linkers described herein may be used to prepare oligonucleotides attached to solid supports, including plastic slides, glass slides, controlled pore glass (CPG), long chain controlled pore glass (LC CPG), and the like. The solid supports are generally preliminarily modified with chemical groups capable of reacting with a functional group on the 3′-aminoalkyl modifier, such as an amino function on the solid support reacting with an activated carboxylic group or carbonyl function on the 3′-aminoalkyl modifier or an activated carboxylic group or carbonyl function on the solid support reacting with an amino function on the 3′-aminoalkyl modifier. The linkers disclosed herein may also be used to synthesize oligonucleotides on solid supports. The linkers disclosed herein may also be used to attach a variety of labeling reagents including fluorescent dyes, biotin and the like, onto oligonucleotides.

A linker is disclosed that includes a compound of the formula: 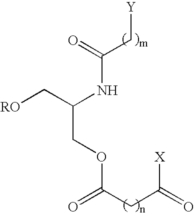
wherein R is selected from the group consisting of hydrogen and an oxygen protecting group, m and n are integers independently selected from the group consisting of 1, 2, 3, 4, 5, 6, 7, and 8; X is an optionally substituted first heteroatom; and Y is an optionally substituted second heteroatom.

A linker that includes a compound of the formula: 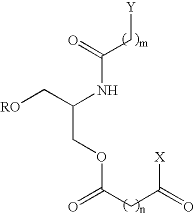
wherein R is selected from the group consisting of hydrogen and an oxygen protecting group, m and n are integers independently selected from the group consisting of 1, 2, 3, 4, 5, 6, 7, and 8; X is an optionally substituted first heteroatom; and Y is an optionally substituted second heteroatom.

In the linker Y may be an optionally substituted nitrogen or an optionally protected nitrogen. X may also be a substituted heteroatom, where at least one of the substituents comprises a solid support. X may also be a substituted nitrogen, where at least one of the substituents comprises a solid support. Y may be a substituted nitrogen, where at least one of the substituents comprises a solid support. Y may be a substituted nitrogen, where at least one of the substituents is selected from the group consisting of diagnostic agents, fluorescent agents, and radioactive agents.

The solid support may be an insoluble silica support, controlled pore glass, long chain controlled pore glass, a glass or a plastic slide or a gel.

An oligonucleotide is disclosed that includes a compound of the formula: 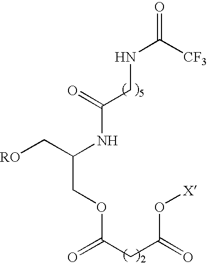
wherein R is dimethoxytrityl; and X′ is succinimid-N-yl.

An oligonucleotide linker is disclosed of the formula: 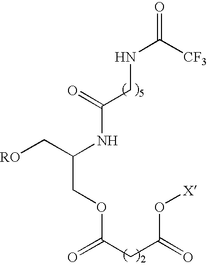
wherein R is dimethoxytrityl; and X′ comprises an insoluble silica support.

The insoluble silica support may be controlled pore glass, long chain controlled pore glass or a glass or plastic slide.

An oligonucleotide conjugate of the formula: 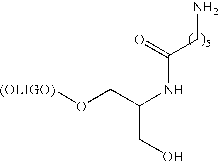
is disclosed wherein “OLIGO” is an oligonucleotide coupled at the 3′-end.

A method for preparing an aminopolyol linker, includes the steps of:
(a) protecting a first hydroxyl group of an aminopolyol by reacting the first hydroxyl group with a compound of the formula R—L, where R is an oxygen protecting group, and L is a leaving group;
(b) acylating the amine of the hydroxyl protected aminopolyol; and
(c) acylating a second hydroxyl group of the aminopolyol.
   The protecting step includes protecting a first hydroxyl group of serinol.

A method for preparing the compound 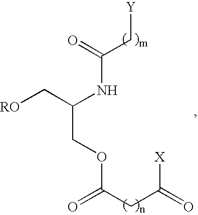
includes the steps of:
(a) protecting a first hydroxyl group of serinol by reacting the first hydroxyl group with a compound of the formula R—L′, where R is an oxygen protecting group, and L′ is a leaving group;
(b) acylating the amine of serinol by reacting the amine with a compound of the formula Y—(CH₂)ₘ—C(O)—L², where L² is a second leaving group; and
(c) acylating a second hydroxyl group of serinol by:
   (1) reacting the second hydroxyl group with a compound of the formula X—C(O)—(CH₂)ₙ—C(O)—L³, where L³ is a third leaving group; or
   (2) reacting the second hydroxyl group with an anhydride of the formula: 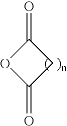
      and reacting the resulting product with a compound capable of forming an activated ester derivative.

The protecting step (a) includes reacting the first hydroxyl group with DMTr—Cl. The acylating step (b) includes acylating the amine with N-hydroxysuccinimid-O-yl 6-(N-trifluoroacetylamino)caproate. The acylating step (c) may also include acylating the second hydroxyl group with succinic anhydride and reacting the resulting product with N-hydroxysuccinimide and an amide coupling agent.

A method for preparing a linker includes the steps of:
(a) protecting a first hydroxyl group of serinol by reacting the first hydroxyl group with a compound of the formula R—L′, where R is an oxygen protecting group, and L¹ is a leaving group;
(b) acylating the amine of serinol by reacting the amine with a compound of the formula Y—(CH₂)ₘ—C(O)—L², where L² is a second leaving group;
(c) acylating a second hydroxyl group of serinol by reacting the second hydroxyl group with a cyclic anhydride; and
(d) reacting the product from step (c) with a compound capable of forming an activated ester derivative with the product of step (c).
(e) reacting the product from step (d) with the solid support.
   The reacting step may be reacting the product from step (d) with controlled pore glass.

A method for fabricating a support with 3′-aminomodified oligonucleotides, includes the steps of:
(a) obtaining one or more aminomodifiers according to claim 5;
(b) coupling one or more oligonucleotides to the one or more aminomodifiers to form one or more oligonucleotide-aminomodifier conjugates; and
(c) coupling the one or more oligonucleotide-aminomodifier conjugates to the support.

The support may be a glass, matrix, gel pads, or plastic. The “one or more oligonucleotides” have a length in the range from about 6 to about 100 nucleotides, or from about 10 to about 100 nucleotides.

### Definitions and Abbreviations

Aminomodifier: a chemical that provides at least one functional amino group to a molecule such as a oligonucleotide and can be used to immobilize the molecule on a support or to introduce a variety of further modifications, e.g. fluorescent dyes, biotin.

Array, microarray: molecules connected to a support in a predetermined arrangement relative to each other. Also known as a chip, DNA chip, DNA microarray, DNA array, microchip, peptide chip or peptide array; includes array of biological molecules such as DNA fragments, peptides, proteins.

Hybridization: the formation of duplex molecules from complementary single strands (e.g., DNA-DNA, DNA-RNA, RNA-RNA). One single stranded nucleic acid molecule is generally labeled, e.g. with a detectable dye (radioactive or fluorescent) and used as a probe that may anneal to molecules with similar sequences that are single stranded. Conditions are varied to detect degrees of similarity, i.e. the more stringent the conditions, the greater the complementarity needed for hybridization to occur.

Nucleic acids : genetic material including single and double stranded deoxyribonucleic acids (DNA), ribonucleoic acids (RNA) and also DNA-RNA hybrids.

Oligomer or oligonucleotide: A nucleotide sequence (DNA or RNA) having about 6 or more nucleotides, and illustratively in the range from about 6 to about 100 nucleotides.

Support: a glass slide, silicon, gold slide, gel pad, acrylamide matrix, or other similar structure on which an array or a microarray of molecules is formed; contains functional groups to attach biomolecules to the support.

Tethering: the manner of immobilization of biomolecules on a support.

Linker: polyfunctional molecule optionally connected to a support; containing functional groups that may provide oligomer chain elongation during solid phase oligonucleotide synthesis; and providing a free amino functional group for post synthesis treatment procedures.

CPG—control pore glass.

LC CPG—long chain controlled pore glass.

DMTr—dimethoxytrityl protective group.

Tfa—trifluoroacetyl protective group.

NOSH—N-hydroxysuccinimide.

PAAG—polyacrylamide gel.

SSPE—saline-sodium phosphate—EDTA buffer.

EDTA—ethylenediamine tetraacetic acid.

Triton X-100—polyethyleneglycol (n=9,10), Octylphenoxypolyethoxyethanol.

HEPES—N-2-hydroxyethylpiperazine-N′-2-ethanesulphonic acid.

UV—ultraviolet.

HPLC—high pressure liquid chromatography.

Phosphoramidite—phosphoramide derivatives of nucleosides used in chemical solid phase oligonucleotide synthesis.

### BRIEF DESCRIPTION OF THE DRAWINGS

FIG. 1 shows the chemical synthesis of a 3′-aminomodifier (5), wherein step (a) is CF₃COOC₂H₅/MeOH; (b) is DMTrCl/pyridine; (c) is NH₄OH/ MeOH; (d) is O-[N-(6-trifluoroacetylamino)caproyl]-N-hydroxysuccinimide/THF (ONOS═N-hydroxysuccinimidyl); (e) is (1) succinic anhydride/pyridine then (2) N-hydroxysuccinimide in the presence of dicyclohexylcarbodiimide/acetone; and (f) is LC CPG/CH₃CN.

FIG. 2 shows detailed structures of 3′-aminomodifier CPG and pathway for creation of 3′-aminoalkylated oligonucleotide (6) during solid phase oligonucleotide synthesis.

FIG. 3 shows the yield (in optical units, OU) of a 3′-aminomodified oligonucleotide (AGTCTCGATCGA-amino) synthesized with the 3′-aminomodifier CPG disclosed herein compared to a conventional 3′-aminomodifier CPG (Glen Research Corp.).

FIG. 4 shows a comparison of hybridization signals from (A) 12-nucleotide residues (“12-mer”) and (B) 20-nucleotide residues (“20-mer”) oligomers prepared with claimed 3′-aminomodifier CPG and a commercially available 3′-aminomodifier CPG (Glen Research Corp.) and immobilized on 3D gel pads after 1 hour hybridization with complementary oligonucleotides labeled with 350 and 35 fmoles of Texas Red dye, respectively.

FIG. 5 shows a comparison of the hybridization signals observed after 24 hours hybridization from (A) 12-nucleotide residues (“12-mer”) and (B) 20-nucleotide residues (“20-mer”) aminomodified oligomers labeled with 350 and 35 fmoles of Texas Red dye, respectively, prepared with claimed and a commercially available 3′-aminomodifier CPG (Glen Research Corp.).

### DETAILED DESCRIPTION OF THE DISCLOSURE

Methods and compositions are described for producing linkers for oligonucleotides, including 3′-aminomodifiers. The linkers described herein may be used to synthesize oligonucleotides with amino-containing groups at their 3′ termini. 3′-terminal aminomodified oligonucleotides can be attached to different kinds of supports to form microarrays. In addition, labels and diagnostic agents, including but not limited to fluorescent chromophores, biotin, and the like can be linked to the oligonucleotides through the terminal amino groups.

In one embodiment, 3′-aminoalkyl modification of oligonucleotides is produced during automatic chemical synthesis of oligonucleotides by using 3′ aminomodifier CPG as a solid phase support.

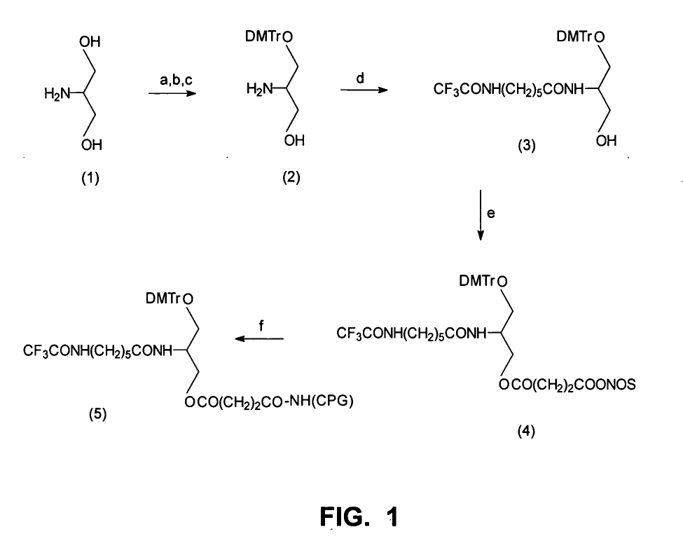

The linker (compound 4 in FIG. 1) represents a polyfunctional moiety, which includes:
1) an activated carboxylic function capable of attaching the linker to any of a variety of solid supports containing free amino functions on the surface and capable of use in solid-phase oligonucleotide synthesis (in the embodiment controlled pore glass, CPG);
2) a protected (in the embodiment DMTr-protected) hydroxylic function that can be deprotected and used for elongation of the oligonucleotide or peptide chain during solid-phase synthesis;
3) a protected (in the embodiment Tfa-protected) amino function that can be deprotected and used for the attachment of an aminated oligonucleotide to the different kinds of appropriately modified surfaces or further modified with imaging agents, diagnostic agents, etc.

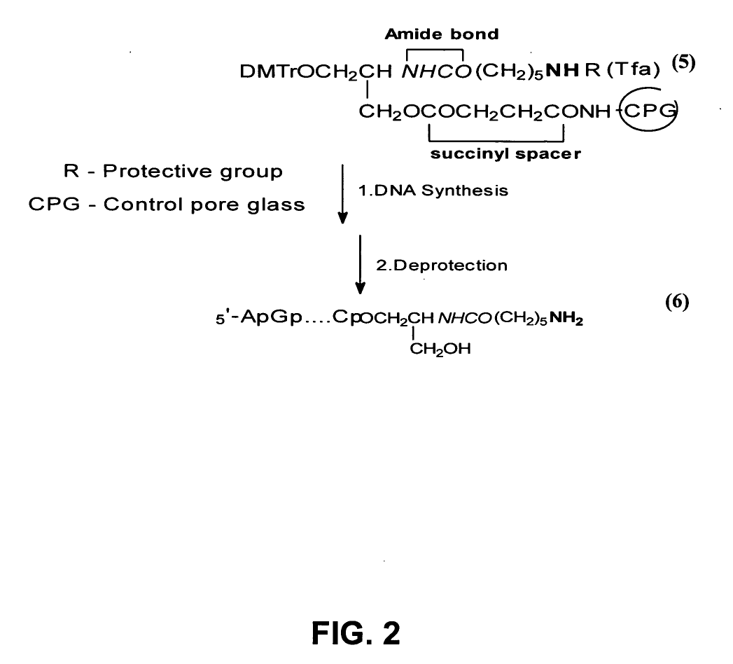

An embodiment of the linkers described herein is 3′-aminomodifier CPG (5), shown in FIG. 1. A scheme for the chemical synthesis of aminomodified oligonucleotides (6) using linker (5) is shown in FIG. 2.

The first step of the synthesis consists in selective protecting of one of the hydroxylic functions in serinol (1) with a DMTr-protective group (or any other acid-labile protective group) with preliminary blocking of the amino function with a Tfa-protective group (or any other base-labile protective group) to obtain compound (2). Alternate acid-labile protective groups and base-labile protective group are generally described in Greene and Wuts (1999). It is appreciated that this chemical transformnation may be carried out as a one-flask procedure using the following steps:
a) reacting serinol with ethyl trifluoroacetate in any of a variety of solvents including methanol, ethanol, iso-propanol, dimethylformamide, and the like, to protect the amino group followed by evaporation of the reaction mixture in vacuo;
b) reacting the resulting product without purification with dimethoxytrityl chloride in pyridine and like solvents to protect one of the hydroxylic function of serinol followed by evaporation of the reaction mixture in vacuo; and
c) reacting the resulting product without purification with methylamine in methanol or with other basic reagent like ammonium hydroxide with the formation of compound (2).

On the next step derivative (2) is reacted with an activated ester like an N-hydroxysuccinimidyl, 4-nitrophenyl, pentachlorophenyl, or like esters of N-protected amino acid to form substance (3) in any variety of organic solvents like acetone, THF, dioxane, pyridine, alcohols. It is appreciated that using such activated esters provides the selective blocking of the amino function in the derivative (2) in the presence of the unprotected hydroxylic function.

A second unprotected hydroxyl functional group of serinol derivative (3) is used for attachment of the linker through a spacer group (illustratively the succinic acid derivative shown in FIG. 2 to a support such as controlled pore glass (CPG) or long chain controlled pore glass (LC CPG) or any other suitable support. This attachment is achieved by acylating compound (3) with an anhydride, such as succinic anhydride, in a suitable solvent, such as pyridine. Alternatively, compound (3) can be acylated with anhydrides of other dicarboxylic acids, like malonic, glutaric, maleic and other diacids. The resulting intermediate ester-acid is converted into an activated ester (4), such as by reaction with N-hydroxysuccinimide, HOBt, and the like, in the presence of a peptide coupling reagent. It is appreciated any of a wide variety of active esters may be prepared in addition to succinimidyloxy esters, including acid chlorides, trimethylsilyl esters, pentafluorophenyl esters, mixed anhydrides, and the like. It is understood that in embodiments where purification of the intermediate represented by compound (4) is desired, the N-hydroxysuccinimidyl activated esters can be used and may be purified before proceeding in the preparation of 3′-modifier CPG. The reaction of (4) with a solid support, such as CPG, LC CPG, and the like in the presence of a solvent, including but not limited to CH₃CN, THF, dioxane, and the like gives 3′-aminomodifier CPG (5). Illustratively, the 3′ aminomodifier CPG has a loading capacity from about 50 to about 55 mkmol of linker (4) immobilized on 1 g of CPG or LC CPG. Then the modified CPG treated with a capping agent, such as pyridine-acetic anhydride-N-methylimidazole mixture. Finally, 3′-aminomodifier CPG (5) is used to start oligonucleotide synthesis by removing the DMT protecting group upon treatment with a standard deblocking solution (3% dichloroacetic acid in dichloromethane).

It is appreciated that the synthesis and process detailed in FIG. 1 for preparing protected derivatives of serinol is equally applicable to other aminopolyols, including but not limited to 3-aminobutan-1,4-diol, 3-aminopentan-1,5-diol, and the like. In any case, the resulting linker system may subsequently be used to synthesize an oligonuclotide on the solid support.

Oligonucleotide synthesis may be carried out on the 3′-aminomodifier (5) disclosed herein, such as the CPG support attached with the linker (4) described in FIG. 1 using a standard automated oligonucleotide synthesizers like Applied Biosystems DNA/RNA synthesizers or Liquid Chemical Dispensing Robot (LCDR).

After the oligonucleotides are synthesized, post processing treatment includes cleaving synthesized oligonucleotides from the CPG supports with simultaneous elution using a 30% ammonium hydroxide solution; deprotection in 30% ammonium hydroxide solution; and followed by evaporation on a standard vacuum-controlled centrifuge. Further processing includes HPLC purification of the synthesized oligonucleotides by Reverse Phase HPLC, followed by evaporation of the HPLC eluates on a standard vacuum-controlled centrifuge, such as the centrifuges of Labconco CentriVap (Labconco Corporation, Kansas City, Mo.).

In the processes disclosed herein, the nucleotide monomers (standard phosphoramidites) are generally protected at the 5′ position. Upon complete synthesis, the final 5′-protecting group is removed by treatment of the evaporated oligonucleotides with an 80% acetic acid solution for 5 min at 25° C., followed by evaporation on a standard vacuum-controlled centrifuge and precipitation of the deprotected oligonucleotides with iso-propanol.

In another embodiment, the aminomodified oligonucleotides are prepared for immobilization on gels, such as acrylamide micro-matrices, and the like, in a 96-well format. The oligonucleotides are evaporated and a final preparation of oligonucleotide solutions in MilliQ-water, in concentrations appropriate for immobilization on the acrylamide micro-matrices, is carried out.

### EXAMPLES

### Example 1.

### Chemical Synthesis of 3′-Aminomodifier CPG 500 Å

### A. 1-O-(4,4′-Dimethoxytrityl)-2-amino-1,3-propanediol (2)

Triethylamine (2.77 ml, 20 mmol) and ethyltrifluoroacetate (3.0 ml, 25 mmol) were added to a suspension of serinol (1) (1.82 g, 20 mmol) in 50 ml of methanol.), The resulting mixture was stirred at room temperature for 40 h and evaporated. The residue was dried by co-evaporation with anhydrous pyridine and dissolved in 40 ml of dry pyridine, to which 4,4′-dimethoxytrityl chloride (7.4 g, 22 mmol) was added. The resulting mixture was stirred overnight at room temperature, evaporated in vacuo, and dissolved in ethyl acetate (100 ml). The solution was sequentially washed with saturated aqueous solution of NaHCO₃ (2×50 ml) and water, dried over Na₂SO₄, and evaporated in vacuo. The residue was dissolved in 40 ml of methanol and then treated with 40% aqueous methylamine for 1 h at room temperature, and is then evaporated. Purification by silica gel column chromatography (8:1 chloroform/methanol) gave 2 (5.0 g, 64% yield); C₂₄H₂₇NO₄; MS (m+H)⁺ 394.5 (calculated: 393.2).

### B. N-(N-(Trifluoroacetyl)-6-aminocaproyl)-1-O-(4,4′-dimethoxytrityl)-2-amino-1,3-propanediol (3)

Serinol derivative 2 (3.93 g, 10 mmol) and the N-hydroxysuccinimide ester of 6-aminocaproic acid (3.56 g, 11 mmol) were dissolved in THF (25 ml), and stirred for 1.5 h at room temperature. The mixture was evaporated in vacuum, diluted with chloroformn (50 ml), and washed with aqueous 5% solution of NaHCO₃. The organic layer was dried over Na₂SO₄, filtered, and concentrated to dryness. The residue was purified by silica gel chromatography (chloroform with a 1-5% MeOH gradient) to give 3 (4.5 g, 75% yield); C₃₂H₃₇F₃N₂O₆; MS (m+H)+ 603.1 (calculated: 602.2).

### C. 1-O-(4,4′-Dimethoxytrityl)-2-N-(6-Trifluoroacetylaminocaproyl)-3-O-(N-hydroxysuccinimidyl-O-succinyl]-2-amino-1,3-propanediol (4)

Succinic anhydride (0.6 g, 6 mmol) was added to a solution of 3 (1.8 g, 3 mmol) in dry pyridine. The reaction mixture was stirred at room temperature overnight, then stopped with methanol (2 ml), and evaporated in vacuo. The oil residue was dissolved in chloroform (25 ml) and washed sequentially with saturated aqueous solution of NaHCO₃ (2×20 ml) and water, dried over Na₂SO₄, evaporated in vacuo, resuspended in acetone, and coevaporated with acetone (2 times). The product was dissolved in acetone (35 ml), and N,N′-dicyclohexylcarbodiimide (619 mg, 3 mmol) was added to the solution. After 5 minutes N-hydroxysuccinimide (380 mg, 3.3 mmol) was added to the solution, and the reaction mixture was stirred at room temperature overnight, and subsequently kept in a refrigerator for 3-4 h. The resulting solid precipitate was filtered through a glass filter, and washed by cold acetone. The combined solution was evaporated in vacuo, and the residue was dissolved in ethyl acetate (30 ml) and washed sequentially with saturated aqueous solution of NaHCO₃ (2×20 ml) and water, dried over Na₂SO₄, and evaporated in vacuo. Purification by silica gel column chromatography (chloroform with a 1-5% MeOH gradient) gave 4 (1.74 g, 73% yield); C₄₀H₄₄F₃N₃O₁₁; MS 799.9 (calculated: 799.28).

### D. 3′-aminomodifier CPG (5)

Controlled pore glass (CPG) 500 Å (LCA CPG 500 A, CPG, Inc., Cat No. LCA00500A, 13.0 g) was suspended in 50 ml of CH₃CN and degassed for 3-5 minutes under vacuo. Compound 4 (990 mg, 1.3 mmol) was dissolved in 9:1 CH₃CN/pyridine (10 ml) and added to the degassed suspension of CPG in CH₃CN. The resulting mixture was kept on a mechanical shaker for 4-6 h at 40° C. The resulting modified CPG was collected on glass filter, washed with 25 ml of 9:1 CH₃CN/pyridine, and then with CH₃CN (2×15 ml). The modified CPG was treated on the filter with a capping mixture of 5:3:0.3 pyridine/acetic anhydride/N-methylimidazole (20 ml), sequentially washed with pyridine (2×15 ml) and acetone (2×15 ml), and then dried in vacuo. The functionalization of the resulting 3′-aminomodified CPG 500 Å was determined by DMTr-deprotection test (see Roland et al., 2001) and was found to be 48.0-54.0 mkmol/g of CPG.

### Example 2

### Chemical Synthesis of 3′-Aminomodified Oligonucleotides (6) Through a Liquid Chemical Dispensing Robot (Standard Protocol)

Oligonucleotide sequence information, such as the sequence AGTCTCGATCGA, for a 96-well plate was entered into a computer controlled Liquid Chemical Dispensing Robot (LCDR). The loading information included parameters of reaction cycles in the LCDR Computer such as injection times for reagent valves; wait times for each step of synthesis; wash cycles after each step; and valve assignment for each reagent.

Reagent bottles were loaded with 0.075 M solutions of nucleotide phosphoramidites in CH₃CN; 0.45 M solution of tetrazole in CH₃CN; Capping mixture A (Ac₂O-lutidine-THF, v/v 10:10:80); Capping mixture B (N-methylimidazole-THF, v/v 10:90); Deblocking solution (3% dichloroacetic acid in CH₂Cl₂); Oxidizing solution (0.1 M iodine in THF-pyridine-water, v/v 60:20:10); and CH₃CN.

The 96-well-plate was filled with 3′-aminomodified controlled pore glass support (CPG) 5, and the plate was set-up for automated chemical synthesis of aminomodified oligonucleotides attached to the solid support.

After oligonucleotides attached to the solid support were synthesized, post processing treatment included: cleaving the synthesized aminomodified oligonucleotides from the CPG support with simultaneous elution using 30% ammonium hydroxide (1 h at 25° C.); deprotection in 30% aqueous ammonium hydroxide (10 h at 25° C.); and evaporation on a standard vacuum-controlled centrifuge.

HPLC purification of synthesized aminomodified oligonucleotides was performed by dissolving the evaporated crude reaction mixtures in 0.05M triethylammonium acetate buffer (1.0 ml, pH 7.0). Reverse Phase chromatography on a Rainin Dynamax HPLC System, model SD 200, US, column C18 (4.6-9.0×250 mm), eluted with a 25-50% gradient of acetonitrile in 0.05 triethylammonium acetate buffer (pH 7.0), 8 min, followed by evaporation of obtained HPLC eluates on a standard vacuum-controlled centrifuge.

Treatment of the evaporated oligonucleotides with 80% acetic acid solution for 5 min at 25° C. to remove the 5′-protecting group was followed by evaporation on a standard vacuum-controlled centrifuge, and precipitation of the deprotected oligonucleotides (6) with isopropanol.

The aminomodified oligonucleotides were then prepared for immobilization on the acrylamide micro-matrices in a 96-well format by dissolving the oligonucleotides in deionized purified MilliQ-water (Millipore, system “Synthesis A10”, US) in 2 mMol concentrations (see generally U.S. Pat. No. 5,552,270). A 100-fold dilution sample was also prepared for UV spectroscopy to calculate the actual amount of oligonucleotides based on the optical density from the UV spectra.

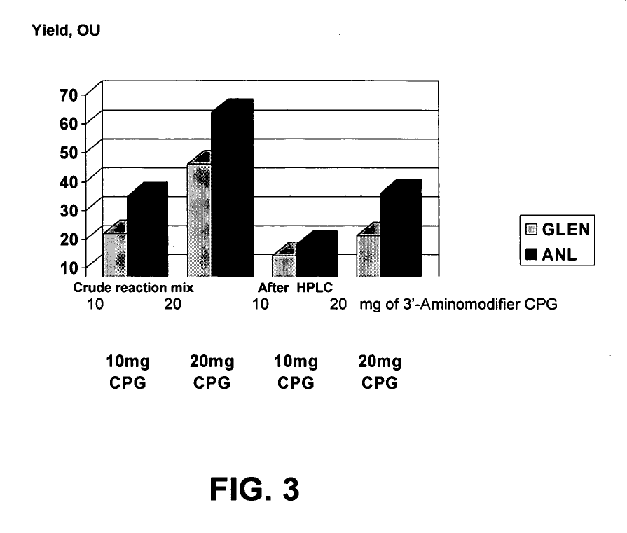

The results of a comparison of the synthetic yields obtained from the 3′-aminomodifier CPG (5) of Example 1 versus a commercially available 3′-aminomodifier CPG (Glen Research) are shown in FIG. 3. The data indicate that the synthetic yield of oligonucleotides prepared with the aminomodifier disclosed herein is comparable to the commercially available aminomodifier.

TABLE 1
Materials and Equipment for Oligonucleotide Synthesis

| Material | Source | Catalog No. |
|---|---|---|
| dmf-dG-CE phosphoramidite | Glen Research | 10-1020-10 |
| Ac-dC-CE phosphoramidite | | 10-1015-10 |
| dT-CE phosphoramidite | | 10-1030-10 |
| dA-CE phosphoramidite | | 10-1000-10 |
| Activator | Glen Research | 30-3100-52 |
| Liquid Chemical Dispensing Robot | Avantech | P98363 |
| HPLC system | Rainin | 9920-113 |
| HPLC-column C18, 4.6 × 250 mm | Supelco | 58355-U |
| | Varian | CP29519 |
| 9.4 × 250 mm | Zorbax | 880975.202 |
| CentriVap | Labconco | U28652-00 |
| Centrifuge | Eppendorf | 5415C |
| 96 well plate | BioLogical Brand | p9605 |
| Eppendorf microcentrifuge tubes, | Fisher | 05-402-24B |
| 1.5 ml Syringe filter, 0.2 μm pore size | Aldrich | Z25994-2 |

### Example 3

### Use of 3′-Aminomodified Oligonucleotides

The primary amino group provided by 3′-aminomodifier described herein may be used to attach to 3′ aminated oligonucleotide various labels, including fluorescent dyes, or to introduce further modifications by a variety of coupling reactions, including amine reactive molecules with active group such as N-hydroxysuccinimide esters of carboxylic function that can couple with the primary amino group. The amino modified oligonucleotides can also be used for the immobilization of the desired oligonucleotide on the different kinds of supports to make an oligonucleotide-based microarrays. Amino modified oligonucleotides are resistant to endonuclease action and therefore are more stable than unmodified oligonucleotides.

The 3′-aminomodified oligonucleotides (6) prepared as described in Example 2, were dissolved in MilliQ water in 2 mMol concentration and further used for immobilization on the acrylamide micro-matrices (3D biochip) as described in U.S. Pat. No. 5,552,270.

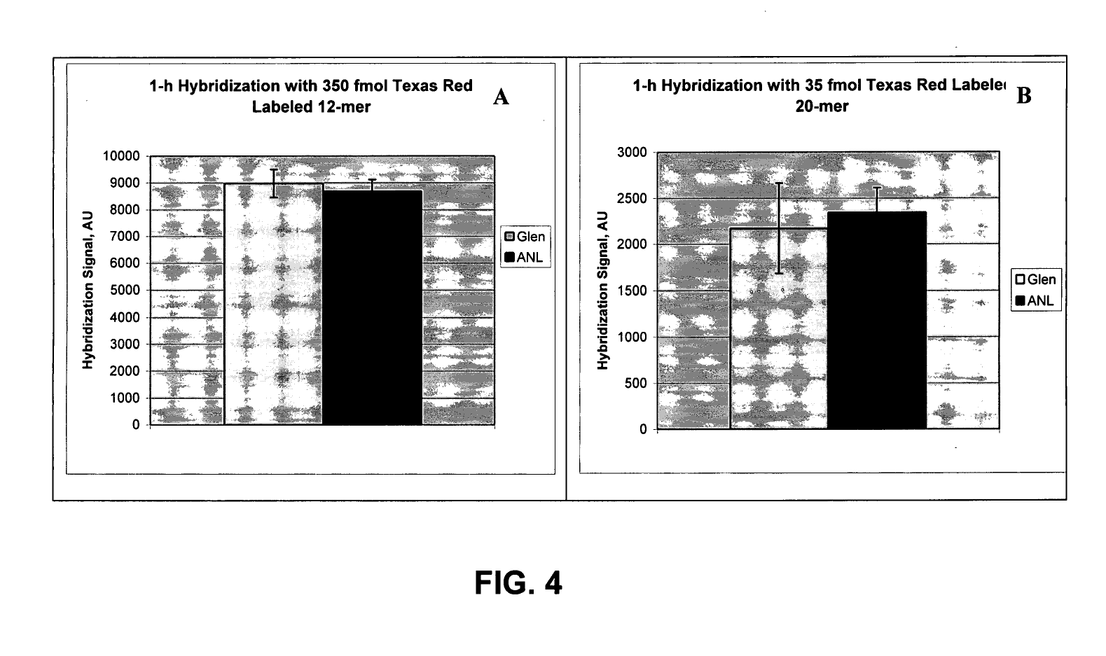

Hybridization signals from 12-mer oligonucleotide and 20-mer oligonucleotide synthesized on 3′ aminomodifier CPG described in Example 2 and on commercial 3′ Aminomodifier CPG (Glen Research Corp.) after 1 h hybridization with complementary oligonucleotides labeled with Texas Red were analyzed (FIG. 4). A biochip containing 12- and 20-mer oligonucleotides with different amino linkers (10 replicates for each probe) was hybridized with a mixture containing complimentary 12-mer (17.5 fmol/μl) and 20-mer (1.75 fmol/μl) oligonucleotides respectively, labeled with Texas Red dye. Hybridization was carried out at room temperature in a 20-μl incubation chamber (Grace Biolabs, Bend, Oreg.) in a buffer containing 1 M guanidine isothiocyanate, 50 mM HEPES (pH 7.5), and 5 mM EDTA. Following the hybridization, the biochip was washed for 5 min 6× SSPE buffer with 0.1% Triton X-100. Then the biochip was briefly rinsed with MilliQ water (water filtered through a 20 μm nylon membrane filter (Millipore, type PTFE, US)) and dried at room temperature. Fluorescent signals from the biochip were acquired on a portable reader (see U.S. Pat. No. 6,620,623). Numerical values of the signals were calculated by a Biochip Imager program (Biochip image grid normalization absolute signal fluorescence measurement using a microscope CCD camera. Copyright University of Chicago, 2001). Average values and standard square deviation of the hybridization signal were calculated for 10 replicates of each probe. “AU” represents arbitrary units.

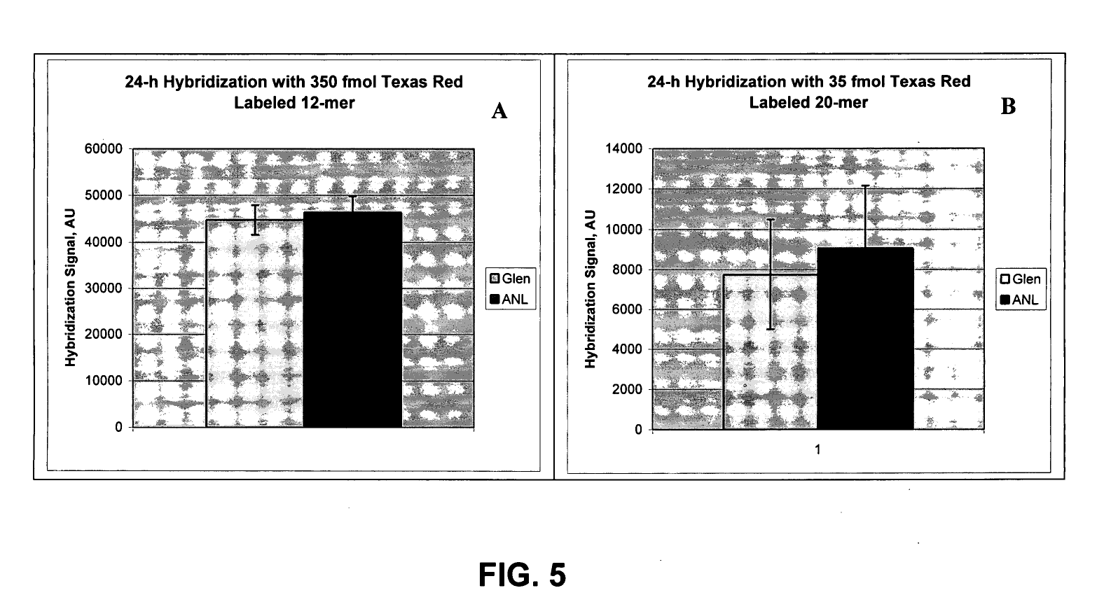

Hybridization signals from 12-mer oligonucleotide and 20-mer oligonucleotide with different linkers after 24-h hybridization with complementary oligonucleotides labeled with Texas Red were analyzed (FIG. 5). Biochips containing 12-mer and 20-mer oligonucleotides (10 replicates for each probe) with different linkers from a commercially available 3′-aminomodifier (Glen Research) and 3′-aminomodifier (5) disclosed herein were hybridized with a mixture containing complementary 12-mer(17.5 fmol/μl) and 20-mer) (1.74 fmol/μl) oligonucleotides labeled with Texas Red dye. Hybridization was carried out at room temperature in a 20 ml incubation chamber (Grace Biolabs, Bend, Oreg.) in a buffer containing 1M guanidine isothiocyanate, 50 mM HEPES (pH 7.5), and 5 mM EDTA. Following the hybridization, the biochip was washed for 5 min with 6× SSPE buffer with 0.1% Triton X-100. Then the biochip was briefly rinsed with MilliQ water and dried at room temperature. Fluorescent signals from the biochip were acquired on a portable reader. Numerical values of the signals were calculated by a Biochip Imager program. Average values and standard square deviation of the hybridization signal were calculated for 10 replicates of each probe. “AU” represents arbitrary units.

Methods for fluorescent labeling of aminoterrninal linker oligonucleotides are known to those of skill in the art (see Haugland, 2002).

### DOCUMENTS CITED

The following documents are incorporated by reference to the extent they relate to protocols used in this disclosure.

Biochip Image Grid Normalization Absolute Signal Fluorescence Measurement Using a Microscopic CCD Camera, Apr. 30, 2001, TXu 993-334, Copyright, University of Chicago.

Greene, T. W., Wuts, P. G. M. (1999) Protective groups in organic chemistry. Third edition. A Wiley-Interscience Publication, New York, USA.

Haugland, R. P., (2002) Handbook of Fluorescent Probes and Research Chemicals, Sections 1.4-1.7, Molecular Probes, Ninth Edition.

Roland, A. et al. (2001) Tetrahedron Letters, 42:3669-3672.

U.S. Pat. No. 5,552,270.

U.S. Pat. No. 6,620,623.

1. A linker comprising a compound of the formula: 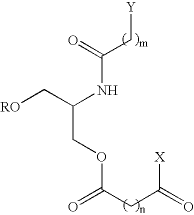
wherein R is selected from the group consisting of hydrogen and an oxygen protecting group, m and n are integers independently selected from the group consisting of 1, 2, 3, 4, 5, 6, 7, and 8; X is an optionally substituted first heteroatom;
and Y is an optionally substituted second heteroatom.

2. A linker comprising a compound of the formula: 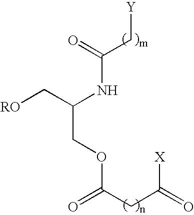
wherein R is selected from the group consisting of hydrogen and an oxygen protecting group, m and n are integers independently selected from the group consisting of 1, 2, 3, 4, 5, 6, 7, and 8; X is an optionally substituted heteroatom; and Y is an optionally substituted nitrogen or an optionally protected nitrogen.

3. The linker of claim 2 wherein X is a substituted heteroatom, where at least one of the substituents comprises a solid support.

4. The linker of claim 2 wherein X is a substituted nitrogen, where at least one of the substituents comprises a solid support.

5. The linker of claim 4 wherein the solid support is an insoluble silica support.

6. The linker of claim 4 wherein the solid support is selected from the group consisting of controlled pore glass, long chain controlled pore glass, glass slides, and plastic slides.

7. The linker of claim 2 wherein Y is a substituted nitrogen, where at least one of the substituents comprises a solid support.

8. The linker of claim 7 wherein the solid support is a gel.

9. The linker of claim 2 wherein Y is a substituted nitrogen, where at least one of the substituents is selected from the group consisting of diagnostic agents, fluorescent agents, and radioactive agents.

10. An oligonucleotide linker comprising a compound of the formula: 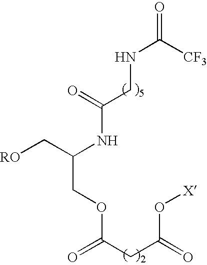
wherein R is dimethoxytrityl; and X′ is succinimid-N-yl.

11. An oligonucleotide linker of the formula: 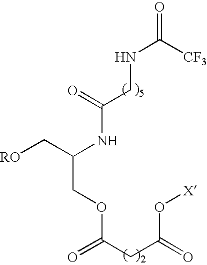
wherein R is dimethoxytrityl; and X′ comprises an insoluble silica support.

12. The olinucleotide linker of claim 11 wherein the insoluble silica support is controlled pore glass, long chain controlled pore glass, and glass slides.

13. An oligonucleotide conjugate of the formula: 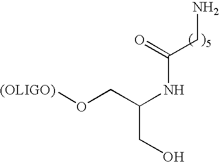
wherein OLIGO is an oligonucleotide coupled at the 3′-end.

14. A method for preparing an aminopolyol linker, the method comprising the steps of:
(d) protecting a first hydroxyl group of an aminopolyol by reacting the first hydroxyl group with a compound of the formula R—L, where R is an oxygen protecting group, and L is a leaving group;
(e) acylating the amine of the hydroxyl protected aminopolyol; and
(f) acylating a second hydroxyl group of the aminopolyol.

15. The method of claim 14 wherein the protecting step includes protecting a first hydroxyl group of serinol.

16. A method for preparing the compound of claim 1, the method comprising the steps of:
(d) protecting a first hydroxyl group of serinol by reacting the first hydroxyl group with a compound of the formula R—L′, where R is an oxygen protecting group, and L¹ is a leaving group;
(e) acylating the amine of serinol by reacting the amine with a compound of the formula Y—(CH₂)ₘ—C(O)—L², where L² is a second leaving group; and
(f) acylating a second hydroxyl group of serinol by:
(1) reacting the second hydroxyl group with a compound of the formula X—C(O)—(CH₂)ₙ—C(O)—L³, where L³ is a third leaving group; or
(2) reacting the second hydroxyl group with an anhydride of the formula: 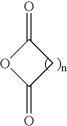
and reacting the resulting product with a compound capable of forming an activated ester derivative.

17. The method of claim 16 wherein the protecting step includes reacting the first hydroxyl group with DMTr—Cl.

18. The method of claim 16 wherein the acylating step (b) includes acylating the amine with N-hydroxysuccinimid-O-yl 6-(N-trifluoroacetylamino)caproate.

19. The method of claim 16 wherein the acylating step (c) includes acylating the second hydroxyl group with succinic anhydride and reacting the resulting product with N-hydroxysuccinimide and an amide coupling agent.

20. A method for preparing the compound of claim 3, the method comprising the steps of:
(f) protecting a first hydroxyl group of serinol by reacting the first hydroxyl group with a compound of the formula R—L¹, where R is an oxygen protecting group, and L¹ is a leaving group;
(g) acylating the amine of serinol by reacting the amine with a compound of the formula Y—(CH₂)ₘ—C(O)—L², where L² is a second leaving group;
(h) acylating a second hydroxyl group of serinol by reacting the second hydroxyl group with a cyclic anhydride; and
(i) reacting the product from step (c) with a compound capable of forming an activated ester derivative with the product of step (c).
(j) reacting the product from step (d) with the solid support.

21. The method of claim 20 wherein the reacting step includes reacting the product from step (d) with controlled pore glass.

22. A method for fabricating a support with 3′-aminomodified oligonucleotides, the method comprising:
(a) obtaining one or more aminomodifiers according to claim 5;
(b) coupling one or more oligonucleotides to the one or more aminomodifiers to form one or more oligonucleotide-aminomodifier conjugates; and
(c) coupling the one or more oligonucleotide-aminomodifier conjugates to the support.

23. The method of claim 22 wherein the support is selected from the group consisting of glass, matrix, gel pads, and plastic.

24. The method of claim 22 wherein the one or more oligonucleotides have a length in the range from about 6 to about 100 nucleotides.

25. The method of claim 24 wherein the oligonucleotides have a length in the range from about 10 to about 100 nucleotides.
